(19) United States
(12) Patent Application Publication
(10) Pub. No.: US 2026/0355416 A1
(43) Pub. Date: Nov. 12, 2026

(54) INFUSION DEVICE WITH SAFETY FEATURES THAT ARE ADJUSTABLE BASED ON CONNECTED EXTERNAL SAFETY DEVICE(S)

(71) Applicant: CareFusion 303, Inc., San Diego, CA (US)

(72) Inventors: Claire Ellen KNIGHT, Arlington, TX (US); Lisa DIGGETT, Overland Park, KS (US); Michael K. Workman, Carlsbad, CA (US)

(21) Appl. No.: 19/120,092

(22) PCT Filed: Oct. 10, 2022
(86) PCT No.: PCT/US2022/046192

§ 371 Date: Apr. 10, 2025

(51) Int. Cl. A61M 5/172; A61M 5/168; A61M 5/36

(52) U.S. Cl. CPC A61M 5/172; A61M 5/16863; A61M 5/365; A61M 2205/18

(57) ABSTRACT

An infusion device includes a safety metric sensor. The infusion device is configured to operate in a default safety mode, where operating in the default safety mode includes monitoring, via the safety metric sensor, an infusion therapy for compliance with a safety metric indicating a property of a fluid path between a fluid container and a fluid administration site. The infusion device is also configured to connect, while operating in the default safety mode, with an external safety device that is configured to monitor the safety metric. Additionally, the infusion device is configured to determine, based on the connection with the external safety device, that the external safety device is monitoring the safety metric. Further, the infusion device is configured to switch, responsive to determining that the external safety device is monitoring the safety metric, from operating in the default safety mode to operating in an adjusted safety mode.

### BACKGROUND

Many infusion therapies involve an external safety device (“ESD”) for monitoring a safety condition, such as air bubbles in an infusion line, particulate contamination, and so on. Oftentimes, the monitored safety condition is also monitored by the infusion pump performing the therapy. This simultaneous monitoring occurs in parallel, with no cooperation or interaction between the ESD and the infusion pump-leading to redundant alarms and wasted resources. Accordingly, there is a need for an infusion pump capable of interfacing with and responding to an ESD.

### SUMMARY

According to various aspects of the subject technology, an infusion device includes a safety metric sensor, a processor, and a non-transitory, computer-readable medium storing instructions that, when executed by the processor, cause the infusion device to operate in a default safety mode. Operating in the default safety mode includes monitoring, via the safety metric sensor, an infusion therapy for compliance with a safety metric indicating a property of a fluid path between a fluid container and a fluid administration site. The instructions also cause the infusion device to connect, while operating in the default safety mode, with an external safety device (“ESD”) that is configured to monitor the safety metric. Additionally, the instructions cause the infusion device to determine, based on the connection with the ESD, that the ESD is monitoring the safety metric. Further, the instructions cause the infusion device to switch, responsive to determining that the ESD is monitoring the safety metric, from operating in the default safety mode to operating in an adjusted safety mode. Operating in the adjusted safety mode includes pausing monitoring the infusion therapy for compliance with the safety metric via the safety metric sensor, of monitoring the infusion therapy for compliance with the safety metric via the ESD.

According to various aspects of the subject technology, a method includes activating an infusion device and a safety metric sensor. The method also includes operating the infusion device in a default safety mode. Operating in the default safety mode comprises monitoring, via the safety metric sensor, an infusion therapy for compliance with a safety metric indicating a property of a fluid path between a fluid container and a fluid administration site. Additionally, the method includes connecting, while operating the infusion device in the default safety mode, the infusion device with an ESD that is configured to monitor the safety metric. Further, the method includes determining, based on the connection with the ESD, that the ESD is monitoring the safety metric. Moreover, the method includes switching the infusion device, responsive to determining that the ESD is monitoring the safety metric, from operating in the default safety mode to operating in an adjusted safety mode. Operating in the adjusted safety mode includes pausing monitoring the infusion therapy for compliance with the safety metric via the safety metric sensor, of monitoring the infusion therapy for compliance with the safety metric via the ESD.

It is understood that other configurations of the subject technology will become readily apparent to those skilled in the art from the following detailed description, wherein various configurations of the subject technology are shown and described by way of illustration. As will be realized, the subject technology is capable of other and different configurations and its several details are capable of modification in various other respects, all without departing from the scope of the subject technology. Accordingly, the drawings and detailed description are to be regarded as illustrative in nature and not as restrictive.

### BRIEF DESCRIPTION OF THE DRAWINGS

For a better understanding of the various described implementations, reference should be made to the Detailed Description below, in conjunction with the Figures. Like reference numerals refer to corresponding parts throughout the Figures and Description.

FIGS. 1A and 1B depict an example patient care system that includes an infusion device and an external safety device (“ESD”), according to aspects of the subject technology.

FIGS. 2A-2C depict example user interfaces that include information relating to the ESD, according to aspects of the subject technology.

FIG. 3 depicts an example process, according to aspects of the subject technology.

FIGS. 4A-4C depict another example process, according to aspects of the subject technology.

FIG. 5 is a conceptual diagram illustrating an example electronic system, according to aspects of the subject technology.

### DETAILED DESCRIPTION

Reference will now be made to implementations, examples of which are illustrated in the accompanying drawings. In the following description, numerous specific details are set forth in order to provide an understanding of the various described implementations. However, it will be apparent to one of ordinary skill in the art that the various described implementations may be practiced without these specific details. In some instances, well-known methods, procedures, components, circuits, and networks have not been described in detail so as not to unnecessarily obscure aspects of the implementations.

As noted above, there is a need for an infusion pump capable of interfacing with and responding to an external safety device (“ESD”). As used herein, an “ESD” is a safety device external to the infusion pump for monitoring a safety condition of an infusion therapy performed by the infusion pump. For example, an ESD might include an air-in-line sensor for monitoring whether too much air is in an infusion line of the infusion pump. Another ESD might include a particulate sensor for detecting whether an infusion fluid flowing through the infusion line is contaminated (e.g., contains too many particulates). An ESD is distinguished from a patient safety device in that patient safety devices monitor safety of an infusion therapy based on physiological measurements of a patient associated with the infusion pump whereas ESDs monitor non-physiological aspects of the infusion, such as monitoring the administration set (e.g., tubing), fluid contents, fluid temperature, and the like. For example, an ESD may measure a safety metric indicating a property of a fluid path between a fluid container and a fluid administration site rather than a specific patient response to fluid administered along the fluid path.

Redundancies arise when an infusion pump and an ESD simultaneously monitor the same safety metric (e.g., air-in-line, particulate contamination, etc.) in isolation. Accordingly, this disclosure relates to the interoperation between infusion pumps and ESDs. For example, when an ESD is monitoring the safety metric of an infusion pump, the infusion pump may be able to adjust its operation to accommodate for the safety monitoring provided by the ESD. The infusion pump may adjust its operation, for example, by including sensor data from the ESD in a safety assessment (e.g., including particulate-in-line data from the ESD in assessing whether an infusion fluid is contaminated) and/or disabling or adjusting a monitoring frequency of other sensors that may be used to monitor similar safety metrics. Or the infusion pump may adjust its operation by disabling detecting, or adjusting the sensitivity of detecting, a safety condition in view of the ESD monitoring the safety condition.

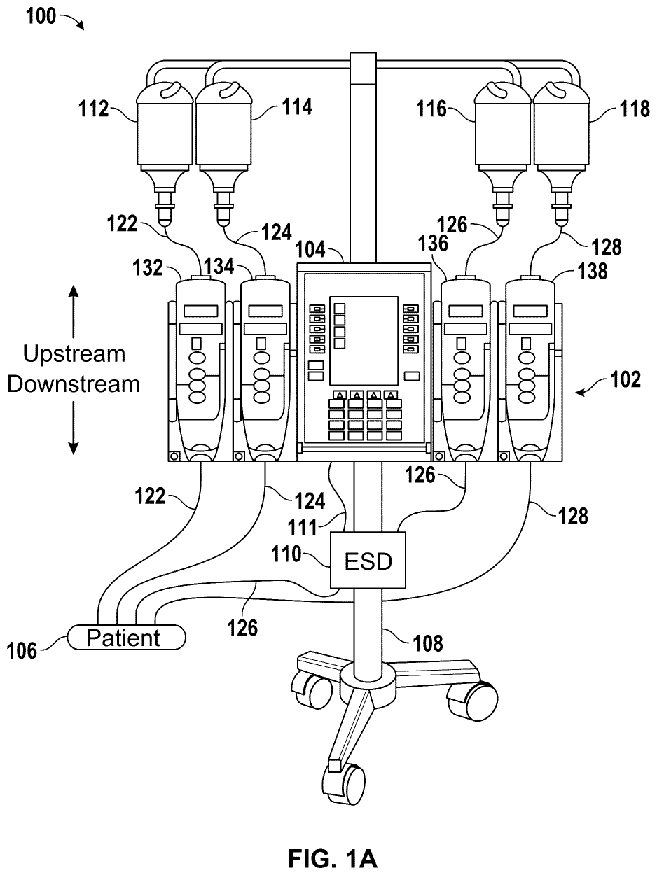

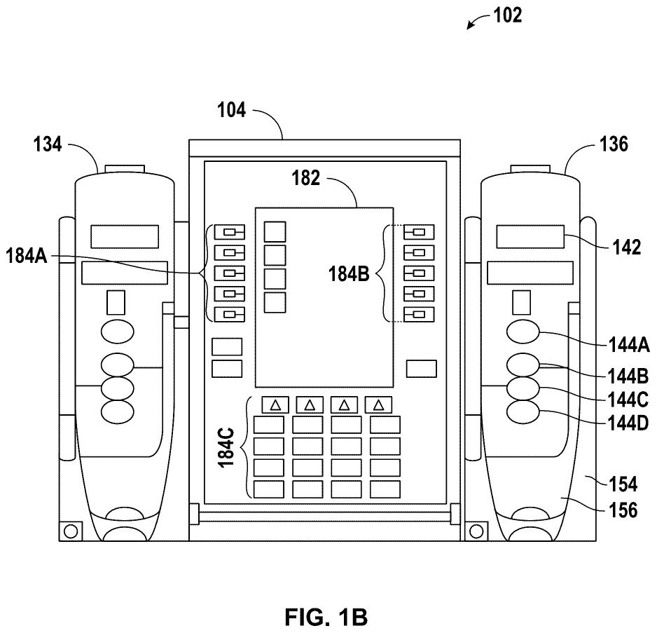

FIGS. 1A and 1B depict an example patient care system 100 that includes an infusion device 102 and an external safety device (“ESD”) 110, according to aspects of the subject technology. Referring first to FIG. 1A, the patient care system 100 includes four fluid infusion pumps 132, 134, 136, and 138, each of which is in operative engagement with a respective fluid administration set 122, 124, 126, and 128. In the depicted example, the infusion pumps are connected to and controlled by a main frame infusion controller 104. Fluid supplies 112, 114, 116, and 118, which may take various forms but in this case are shown as bottles, are inverted and suspended above the pumps. Fluid supplies 112, 114, 116, and 118 may also take the form of bags or other types of containers. Both the infusion device 102 and the fluid supplies 112, 114, 116, and 118 are mounted to a roller stand or pole 108. The specific fluid supplies 112, 114, 116, and 118, as well as their orientation (e.g., mount location, mount height, mounting type, etc.) within the care area, may generate one or more interaction records. The interaction record for a set, for example, may be generated in part by detecting a scannable code associated with the set or detecting a physical structure on the set that encodes identifying information for the set prior to use.

As shown in the example implementation, each administration set 122, 124, 126, and 128 is connects a respective fluid supply 112, 114, 116, and 118 to a patient 106 so that the patient 106 may receive the fluids in all the fluid supplies 112, 114, 116, and 118. The administration set may be identified either actively by, for example, scanning by a clinician or passively by, for example, wireless or optical detection of the administration set.

In the depicted example, a separate infusion pump 132, 134, 136, and 138 is used to infuse each of the fluids of the fluid supplies 112, 114, 116, and 118 into the patient 106. The infusion pumps 132, 134, 136, and 138 are flow control devices that will act on the respective tube or fluid conduit of the fluid administration set to move the fluid from the fluid supply through the conduit and to the patient 106. Because individual infusion pumps 132, 134, 136, and 138 are used, each pump may be individually set to the pumping or operating parameters required for infusing the particular medical fluid from the respective fluid supply into the patient at the particular rate prescribed for that fluid by the clinician.

The example external safety device (“ESD”) 110 depicted in example patient care system 100 may include an air trap, an air detector, a flow detector, a particulate detector, a spectral analyzer, or another device configured to monitor a safety metric associated with the infusion device 102. As depicted, a downstream portion of the administration set 126 passes through the ESD 110. Additionally, or in the alternative, the ESD 110 may be located upstream of the infusion device 102. For example, the ESD 110 may analyze a safety metric (e.g., an air-in-line metric, a flow metric, a particulate-in-line metric, or a spectral metric) of a fluid as it flows through the set 126. A metric may measure a quantity or a quality of a target property, such as air bubbles, flow rate, particulate level, or light. The metric may be measured at intervals or for a time series by sampling values over time. When more than one value is collected for analysis, the metric may reflect an average, a moving average, a maximum for period, a minimum for period, correspondence to a threshold or range, or the like. The ESD 110 is also connected to the infusion device 102 via a wired connection 111. As discussed herein, the infusion device 102 (e.g., the main frame infusion controller 104 or attached infusion pump) may be configured to detect the ESD 110, receive signals from or send signals to the ESD 110, and/or control the ESD 110. Additionally, the infusion device 102 may be configured to adjust its own operation in accordance with the ESD 110. In some implementations, the ESD 110 may connect to the infusion device 102 via a wireless connection (e.g., a Bluetooth connection, a Wi-Fi connection).

Typically, medical fluid administration sets have more parts than are shown in FIG. 1A. Many have check valves, drip chambers, valved ports, connectors, and other devices well known to those skilled in the art. These other devices have not been included in the drawings so as to preserve clarity of illustration.

FIG. 1B offers a closer view of a portion of the infusion device 102 of FIG. 1A, according to various aspects of the subject technology. One or more features of the infusion device 102 may be configured or controlled based on the ESD features described. FIG. 1B shows two of the fluid infusion pumps 134 and 136 mounted at either side of a main frame infusion controller 104, and the displays and control keys of each pump 134 and 136, with the main frame infusion controller 104 being capable of programming each infusion pump 134, 136 (as well as not-pictured infusion pumps 132 and 138). The infusion pump 136 includes a door 154 and a handle 156 that operates to lock the door 154 in a closed position for operation and to unlock and open the door 154 for access to the internal pumping and sensing mechanisms and to load administration sets for the pump 136. When the door 154 is open, the tube can be connected with the pump 136. When the door 154 is closed, the tube is brought into operating engagement with the pumping mechanism, the upstream and downstream pressure sensors, and the other equipment of the pump 136. A display 142, such as an LED display, is located in plain view on the door in this embodiment and may be used to visually communicate various information relevant to the pump 136, such as alert indications (e.g., alarm messages). Control keys 144A-144D exist for programming and controlling operations of the infusion pump as desired. In some implementations, the control keys 144A-144D may be presented as interactive elements on the display 142 (e.g., a touchscreen display). The infusion device 102 and/or the infusion pump 136 may also include audio alert equipment in the form of a speaker (not shown).

The main frame infusion controller 104 of the infusion device 102 includes a display 182 for visually communicating various information, such as the operating parameters of a connected pump and alert indications and alert messages, and control keys 184A-184C for selecting and/or setting control parameters and/or options for controlling the infusion device 102 and connected modules. The main frame infusion controller 104 may also include a speaker to provide audible alerts. In some implementations, the display 182 may be implemented as a touchscreen display. In such implementations, the control keys 184A or 184B may be omitted or reduced in number by providing corresponding interactive elements via a graphical user interface presented via the display 182. In some implementations, each control key 184A-184C may select a corresponding option displayed in the display 182.

The main frame infusion controller 104 may include a communications system (not shown) with which the main frame infusion controller 104 may communicate with external equipment such as a medical facility server or other computer and with a portable processor, such as a handheld communication device or a laptop-type of computer, or other information device that a clinician may have to transfer information as well as to download drug libraries (e.g., to infusion pump 136). The communication module may be used to transfer access and interaction information for clinicians encountering the main frame infusion controller or device coupled therewith (e.g., pump 136, a bar code scanner). The communications system may include one or more of a radio frequency (RF) system, an optical system (e.g., infrared), a Bluetooth system, or other wired or wireless system. The bar code scanner and communications system may alternatively be included integrally with the infusion pump 136, such as in cases where a main frame infusion controller 104 is not used, or in addition to one with the main frame infusion controller 104. Further, information input devices need not be hard-wired to medical instruments, information may be transferred through a wireless connection as well.

Additionally, other types of modules may be connected to the pump modules or to the main frame infusion controller 104 such as a syringe pump module, a patient controlled analgesic module, external safety devices (“ESDs”) 110, a patient safety device such as an end tidal CO2 monitoring module or an oximeter monitoring module, or the like. Some ESDs may include, for example, an air trap, an air detector, a flow detector, a particulate detector, or a spectral analyzer. According to various implementations, the infusion device 102 (e.g., main frame infusion controller 104) may be configured to operate in one of a plurality of modes with regard to whether an ESD is connected to the infusion device 102. For example, the infusion pump 102 may operate in a default safety mode when an ESD 110 is not connected to the infusion pump 102 (see operation 304). However, after successfully pairing or connecting the ESD 110 to the infusion pump 102, the infusion pump 102 may switch to operating in an adjusted safety mode (see operation 310), as discussed in more detail below.

In some embodiments, the pressure measurements from the upstream and/or downstream pressure sensors are transmitted to a server or other coordination device, and the methods disclosed herein are implemented on the server or other coordination device. For example, more sophisticated and computationally intensive approaches like machine-learning can be implemented on the server (or on a PCU with a larger memory and/or CPU resources). In some embodiments, machine learning is used to identify empty conditions based on pressure signals received from the pump.

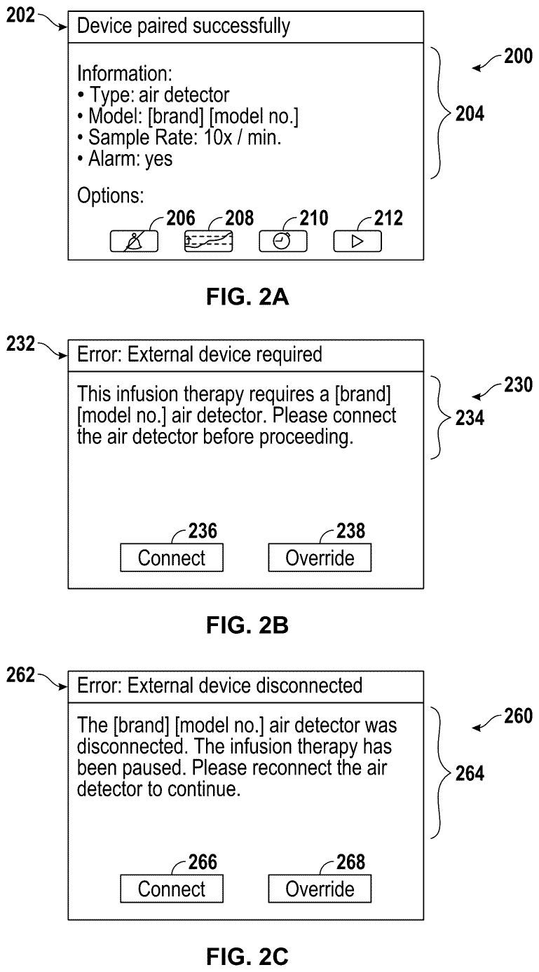

FIGS. 2A-2C depict example user interfaces 200, 230, and 260 that include information relating to a connected ESD 110, according to aspects of the subject technology. In some implementations, the user interfaces 200, 230, and 260 are displayed at the display 182 of the main frame infusion controller 104.

Starting with FIG. 2A, a user interface 200 indicates that a successful connection was established between the infusion device 102 and the ESD 110. The depicted user interface 200 includes a header area 202 configured to display a header notification (e.g., “Device paired successfully”) and an information area 204 configured to display a notification regarding the ESD 110, including the ESD's type (e.g., “air detector”), its sample rate (e.g., “10 x/min.”), and whether it includes an alarm (e.g., “yes”). In some implementations, the information area 204 also displays a particular brand name or model number of the ESD 110, such as for an infusion treatment requiring a specific brand or model of the ESD 110.

The depicted user interface 200 also includes a mute button 206, a threshold button 208, a sample button 210, and a start button 212. In some implementations, the mute button 206, when activated by a user, mutes an alarm of the infusion device. For example, if the infusion device 102 includes a safety metric sensor (e.g., an air-in-line sensor) and the ESD 110 includes the same type of safety metric sensor (e.g., an air-in-line sensor), both the infusion device 102 and the ESD 110 may simultaneously trigger their respective alarms on detecting a hazard associated with the safety metric. Accordingly, the user may wish to mute the alarm of the infusion device 102 by activating mute button 206 (e.g., to avoid a redundant alarm, to avoid the noise of two alarms, to avoid needing to respond to two alarms).

In some implementations, the threshold button 208, when activated by the user, adjusts a safety metric threshold of the infusion device 102. For example, the user may adjust the safety metric threshold to decrease the likelihood of the infusion device alarm triggering in instances where both the infusion device 102 and the ESD 110 include the same type of safety metric sensor. For example, the user may raise the safety metric threshold of the infusion device 102 such that the infusion device 102 does not trigger an alarm for the safety metric unless a higher safety metric threshold is satisfied (e.g., to account for a faulty ESD 110). In some implementations, the threshold may automatically adjust based on connection status with the ESD 110.

In some implementations, the sample button 210, when activated by the user, adjusts a sample rate of the infusion device 102. As with the above examples, if the infusion device 102 and the ESD 110 include safety metric sensors of the same type, the user may wish to decrease the rate at which the infusion device 102 samples or monitors the safety metric (e.g., to decrease a computational load associated with frequent sampling of the safety metric). In some implementations, the sampling rate may automatically adjust based on connection status with the ESD 110.

In some implementations, the start button 212, when activated by the user, begins an infusion therapy. For example, the start button 212 may initiate an infusion therapy at the infusion device 102 (e.g., via infusion pump 136) while operating in an adjusted safety mode (e.g., as programmed by the user via the mute button 206, the threshold button 208, or the sample button 210). After the user selects the start button 212, the connection with the ESD 110 may be reconfirmed (e.g., to ensure the ESD is connected to the infusion device, capable of monitoring a safety metric, capable of triggering an alarm), as is discussed in more detail below.

Referring now to FIG. 2B, a user interface 230 indicates that the ESD 110 is required for a requested infusion therapy. The depicted user interface 230 includes a header area 232 configured to display a header notification (e.g., “Error: External device required”) and an information area 234 configured to display a notification regarding the ESD 110 (e.g., indicating that a particular brand or model of air detector ESD is required).

The depicted user interface 230 also includes a connect button 236 and an override button 238. In some implementations, the connect button 236, when activated by a user, initiates connecting to the ESD 110 (e.g., by displaying a series of connection instructions, by opening a pairing screen). In some implementations, the override button 238, when activated by the user, overrides the ESD requirement. For example, if the ESD 110 is connected to the infusion device 102 but the infusion device 102 does not recognize the connection, the user may override the ESD requirement by selecting the override button 238. As another example, if an ESD is not available the user may override the ESD requirement by activating the override button 238.

In some implementations, the user interface 230 does not include an override button 238. For example, if it is necessary (e.g., medically necessary, legally necessary) to connect an ESD to the infusion pump 102 prior to the infusion therapy, the user interface 230 may not allow the user to initiate the infusion therapy without first connecting an ESD to the infusion pump.

Finally, referring to FIG. 2C, a user interface 260 indicates that the ESD 110 has disconnected. The depicted user interface 260 includes a header area 262 configured to display a header notification (e.g., “Error: External device disconnected”). The user interface 260 also includes an information area 264 configured to display a notification regarding the ESD 110. In the depicted implementation, the notification indicates that the ESD 110 was disconnected, the infusion therapy was paused, and the ESD 110 ought to be reconnected before continuing the therapy.

The user interface 260 also includes a connect button 266 and an override button 268. The connect button 266 and the override button 268 may be similar to the connect button 236 and the override button 238 of the user interface 230. For example, the connect button 266, when activated by a user, may initiate re-connecting to the ESD 110. Further, the override button 268, when activated by the user, may override the ESD requirement or recommendation (e.g., by resuming the infusion therapy). As with the user interface 230, in some implementations, the user interface 260 does not include the override button 268 (e.g., where the ESD 110 is mandatory).

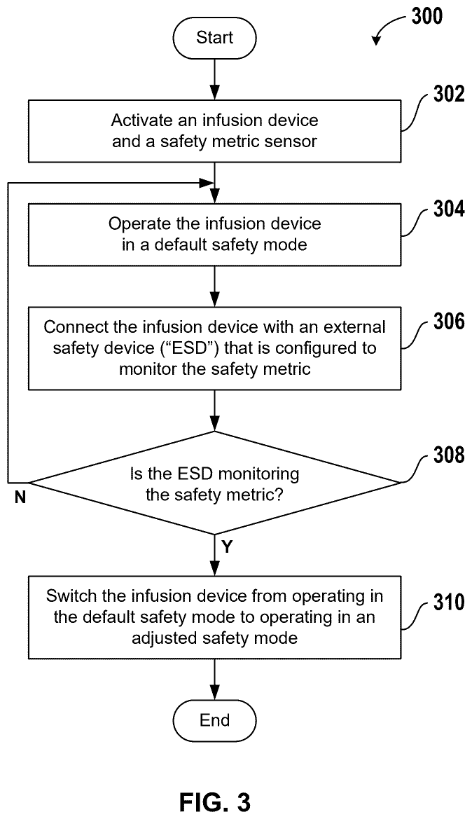

FIG. 3 depicts an example process 300, according to aspects of the subject technology. One or more blocks of process 300 may be implemented, for example, by one or more computing devices, such as infusion device 102. In some implementations, one or more of the blocks may be implemented based on one or more machine learning algorithms. In some implementations, one or more of the blocks may be implemented apart from other blocks, and by one or more different processors or devices. Further, for explanatory purposes, the blocks of example process 300 are described as occurring in serial, or linearly. However, multiple blocks of example process 300 may occur in parallel. Additionally, the blocks of example process 300 need not be performed in the order shown and one or more of the blocks of example process 300 need not be performed.

As depicted, the process 300 includes activating an infusion device (e.g., infusion device 102) and a safety metric sensor (302). The safety metric sensor may include an air trap, an air-in-line sensor, a flow detector, a particulate detector, a spectral detector, or another sensor for monitoring a characteristic of a fluid provided by the infusion device. The infusion device may be configured to control and/or receive data from multiple safety metric sensors, including other safety metric sensors not mentioned herein.

The process 300 also includes operating the infusion device in a default safety mode (304), where operating in the default safety mode includes monitoring, via the safety metric sensor, an infusion therapy for compliance with a safety metric indicating a property of a fluid path (e.g., including administration set 126) between a fluid container (e.g., fluid supply 116) and a fluid administration site (e.g., at patient 106). For example, the safety metric may be an air-in-line metric, and the safety metric sensor may be an air-in-line sensor. Accordingly, monitoring the infusion therapy for compliance with a safety metric may include collecting air-in-line data from the air-in-line sensor and determining whether the air-in-line data complies with an air-in-line metric (e.g., an air-in-line safety threshold).

Additionally, the process 300 includes connecting the infusion device with an ESD that is configured to monitor the safety metric (306). For example, connecting (306) the infusion device with the ESD may include plugging the ESD into the infusion device (e.g., using wired connection 111). As another example, connecting the infusion device with the ESD may include wirelessly connecting the infusion device with the ESD (e.g., via Bluetooth, Wi-Fi). As yet another example, connecting the infusion device with the ESD may include navigating a series of prompts or instructions (see, e.g., user interfaces 200, 230, and 260).

Further, the process 300 includes determining whether the ESD is monitoring the safety metric (308). The determination may be made, for example, by the infusion device 102 or the main frame infusion controller 104. Additionally, the determination may be based on receipt of a message (or contents thereof) from the ESD. In some implementations, the determination may monitor for a message within a specific period of time (e.g., every 5 seconds) or a specific message (e.g., “ESD connected” message). Responsive to determining that the ESD is not monitoring the safety metric (308-N), the process 300 includes operating the infusion device according to the default safety mode (304) (e.g., until a determination is made that the ESD is monitoring the safety metric, until another ESD connects to the infusion device).

However, responsive to determining that the ESD is monitoring the safety metric (308-Y), the process 300 includes switching the infusion device from operating in the default safety mode to operating in an adjusted safety mode (310). The infusion device may not need to monitor the safety metric while the ESD is monitoring the safety metric. In some implementations, operating in the adjusted safety mode includes pausing monitoring of the safety metric by the infusion device. The infusion device may, in some instances, reduce the level at which the safety metric is monitored (e.g., reduce the frequency of activating the sensor, or use a less resource intensive analysis algorithm) while the ESD is monitoring the safety metric. In this regard, resources such as energy and computational power may be saved. The infusion device may, on detecting the presence of an ESD, disable the safety metric sensor from being used by the infusion device.

In some implementations, operating in the adjusted safety mode includes monitoring the infusion therapy for compliance with the safety metric and collecting the safety metric data via the safety metric sensor. Additionally, operating in the adjusted safety mode may include receiving additional safety metric data from the ESD and determining whether the safety metric satisfies a hazard threshold based on the safety metric data and the additional safety metric data. For example, determining that the safety metric satisfies a hazard threshold may include determining that the safety metric data or the additional safety metric data satisfy the hazard threshold. As another example, determining that the safety metric satisfies a hazard threshold may include determining that the safety metric data and the additional safety metric data satisfy a second threshold, different from (e.g., lower than) the hazard threshold.

Responsive to determining that the safety metric satisfies the hazard threshold, in some implementations, the process 300 includes pausing an infusion therapy. For example, pausing the infusion therapy may include displaying a notice indicating that the safety metric satisfies the hazard threshold. Additionally, the notice may include information regarding data (e.g., data from the infusion device, or data from the ESD) used in determining that the safety metric satisfies the hazard threshold (e.g., indicating a hazard was detected by the infusion device or by the ESD). Similarly, responsive to determining that the safety metric satisfies the hazard threshold, in some implementations, the process 300 includes activating a safety mode of the infusion device. For example, the safety mode may include procedures for handling a hazard (e.g., pausing or slowing the infusion therapy, notifying a user, or triggering an alarm).

In some implementations, operating in the default safety mode includes triggering an alarm in response to detecting that the safety metric satisfies a hazard threshold. Accordingly, in some implementations, operating in the adjusted safety mode includes deactivating the alarm. For example, as noted with respect to the mute button 206 of the user interface 200, the infusion device may deactivate its alarm if the ESD also includes an alarm. As another example, the infusion device may automatically deactivate the alarm while operating in the adjusted safety mode. Moreover, in other implementations, operating in the adjusted safety mode includes triggering an alarm in response to detecting that the safety metric satisfies an adjusted hazard threshold that is different from (e.g., higher than) the hazard threshold.

In some implementations, operating in the default safety mode includes causing the safety metric sensor to sample the safety metric at a default sample rate. Accordingly, in some implementations, operating in the adjusted safety mode includes causing the sensor to sample the safety metric at an adjusted sample rate that is different from the default sample rate. For example, the infusion device may not need to sample the safety metric as frequently if the ESD is already sampling the safety metric. This is discussed above in more detail with respect to the sample button 210 of the user interface 200.

In some implementations, the process 300 also includes determining, based on the connection between the infusion device and the ESD, that the ESD is no longer monitoring the safety metric (or that the ESD is no longer connected to the infusion device). Responsive to determining that the ESD is no longer monitoring the safety metric (or connected to the infusion device), the process 300 includes switching the infusion device from operating in the adjusted safety mode to operating in the default safety mode. In addition to switching to operating in the default mode, the process 300 may also include notifying a user that the ESD is no longer monitoring the safety metric (or connected to the infusion device). Alternatively, responsive to determining that the ESD is no longer monitoring the safety metric (or connected to the infusion device), the process 300 may include pausing the infusion therapy or informing a user that the ESD is no longer monitoring the safety metric. As an example of this, see the above discussion with regards to the user interface 260.

In some implementations, the process 300 further includes receiving, at the infusion device, an infusion request indicating an infusion therapy and an ESD requirement that specifies a type of ESD. The process 300 may also include receiving, at the infusion device, a type of the ESD from the ESD. For example, the type of the ESD may indicate a brand or model of the ESD. As another example, the type of the ESD may indicate a safety metric monitoring function of the ESD, such as whether the ESD monitors an air-in-line metric. Accordingly, the process 300 may include determining that the type of the ESD matches the specified type of ESD. Further, the process 300 may include initiating the infusion therapy at the infusion device responsive to determining that the type of the ESD matches the specified type of ESD. For example, some infusion therapies may require a particular type of ESD (e.g., an ESD configured to monitor a particular safety metric, a particular brand or model of ESD).

In some implementations, the ESD includes an air trap, an air detector, a flow detector, a particulate detector, or a spectral analyzer. Accordingly, in some implementations, the safety metric includes an air-in-line metric, a flow metric, a particulate-in-line metric, or a spectral metric. As noted above, the safety metric may measure a quantity or quality of a target property, such as air bubbles, flow rate, particulate level, or light. The metric may be measured at intervals or for a time series by sampling values over time. When more than one value is collected for analysis, the metric may reflect an average, a moving average, a maximum for period, a minimum for period, correspondence to a threshold range, or the link. Further, in some implementations, determining that the ESD is monitoring the safety metric includes receiving, from the ESD, additional safety metric data including air-in-line data, flow data, particulate-in-line data, or spectral data.

According to various implementations, data received from the ESD (e.g., regarding a safety hazard) is provided to a machine learning model that is used to refine the safety metric sensing capabilities of the infusion device. For example, if the ESD recognizes an air-in-line hazard that the infusion device fails to notice, the machine learning model can be used to refine the detection capabilities of the infusion device (e.g., by adjusting a threshold at which an air-in-line hazard is raised).

In some implementations, the improvement may be achieved by combining ESD data with infusion device data. For example, a classifier model may be trained to identify a hazard condition based on historic ESD data and cotemporaneous infusion device data. Once trained, the model may receive a set or series of values of ESD data and contemporaneous infusion device data (e.g., safety metric or measurements derived therefrom) and generate an output indicating a hazard condition. The output may identify a specific hazard condition, a magnitude of the hazard, an alarm level appropriate for the inputs, and/or a confidence value indicating the likelihood that the modeled output reflects the actual infusion state. As another example, the infusion device may alter the hazard detection algorithm used based on connectivity with an ESD. If an ESD is connected, the infusion device may use a first algorithm to detect the hazard based on information from the ESD alone or in combination with data generated by the infusion pump. If an ESD is not connected, the infusion device may use a different algorithm to detect the hazard based on data generated by the infusion pump. In this way, the resources are conserved by leveraging a potentially more sophisticated but resource intensive algorithm when the ESD information is actually available and changing to a different algorithm that may be more resource efficient if the ESD is unavailable.

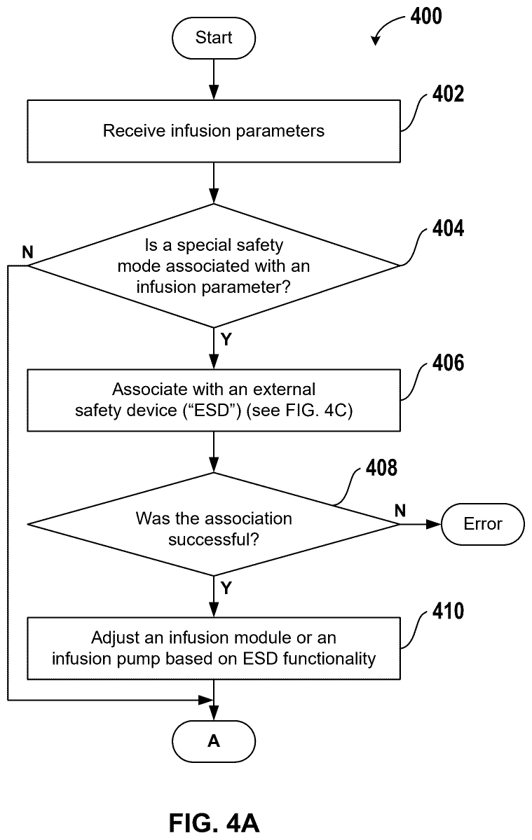

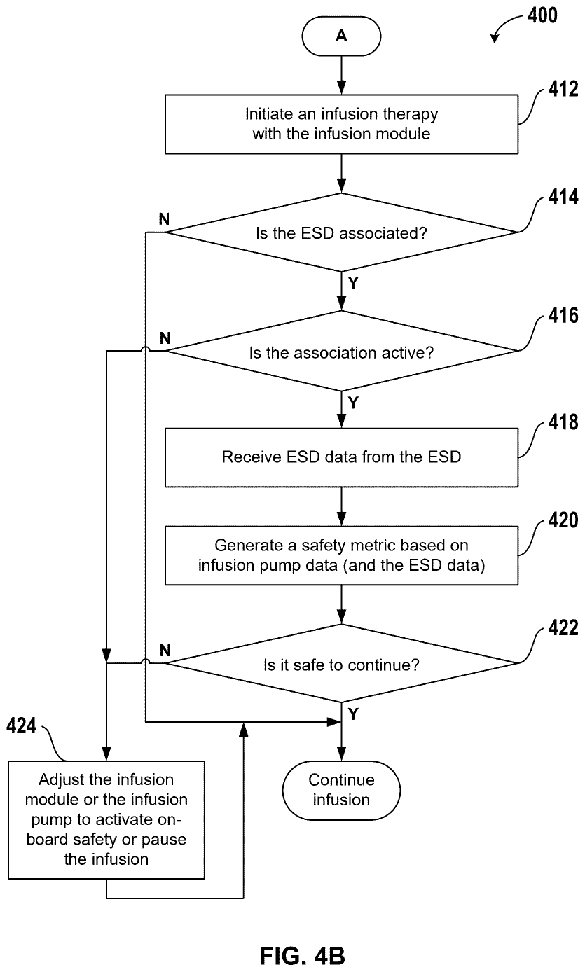

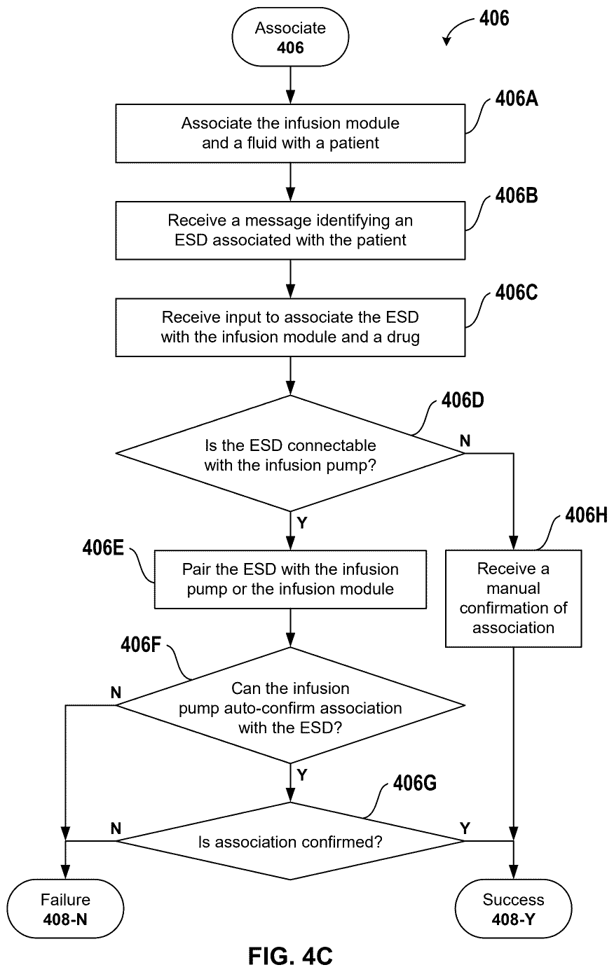

FIGS. 4A-4C depict another example process 400, according to aspects of the subject technology. As with the process 300, one or more blocks of process 400 may be implemented, for example, by one or more computing devices, such as infusion device 102. In some implementations, one or more of the blocks may be implemented based on one or more machine learning algorithms. In some implementations, one or more of the blocks may be implemented apart from other blocks, and by one or more different processors or devices. Further, for explanatory purposes, the blocks of example process 400 are described as occurring in serial, or linearly. However, multiple blocks of example process 400 may occur in parallel. Additionally, the blocks of example process 400 need not be performed in the order shown and one or more of the blocks of example process 400 need not be performed.

As depicted in FIG. 4A, the process 400 includes receiving infusion parameters (402). In some implementations, the infusion parameters are received at an infusion device (e.g., the infusion device 102). For example, the infusion parameters may include a fluid type (e.g., a medication, a saline solution), an IV set type (e.g., specifying an IV set length), a flow rate (e.g., 50 mL/hr), a total volume to be infused (e.g., 400 mL), an ESD requirement (e.g., requiring a particulate sensor ESD), or an ESD recommendation (e.g., recommending an air-in-line ESD).

The process 400 also includes determining whether a special safety mode is associated with an infusion parameter (404). A special safety mode may be associated with an infusion parameter, for example, if the infusion parameter is likely to create a safety hazard. A high infusion flow rate is one example of an infusion parameter with which a special safety mode might be associated. When an infusion pumps are operated at high speeds, the likelihood of air entering the infusion line increases. Accordingly, a special safety mode associated with a high flow rate might include requiring that an ESD (e.g., an air-trap ESD) is connected to the infusion pump prior to starting the infusion therapy. A medication susceptible to decomposing is another example of an infusion parameter with which a special safety mode might be associated. In order to prevent particulate contamination, the special safety mode might require connecting an ESD (e.g., a particulate sensing ESD) to the infusion pump prior to starting the infusion therapy of the fluid.

In some implementations, determining whether a special safety mode is associated with an infusion parameter includes determining whether a value or a potential value of the infusion parameter (e.g., a flow rate) satisfies a threshold (e.g., exceeds a high-flow-rate threshold). In some implementations, determining whether a special safety mode is associated with an infusion parameter includes searching for the infusion parameter (e.g., a medication susceptible to decomposing) in a database and checking whether any programmed special safety mode (e.g., requiring a particulate sensing ESD) is stored in the database and associated with the infusion parameter.

Responsive to determining that a special safety mode is not associated with an infusion parameter (404-N), the process 400 includes initiating an infusion therapy with the infusion module (412). The initiating operation (412) is discussed in more detail below. On the other hand, responsive to determining that a special safety mode (e.g., ESD requirement) is associated with an infusion parameter (404-Y), the process 400 includes associating with an ESD (406). For example, associating with the ESD (406) may include establishing a physical (e.g., wired) or wireless (e.g., Bluetooth, Wi-Fi) connection with the ESD. Further, associating with the ESD (406) may include handshake mode, security measures, or a verification process.

Additionally, the process 400 includes determining whether the association was successful (408). The success of the association may be based on one or more messages received from the ESD by the infusion pump. In some implementations, the messages may be part of a standards-based association protocol, such as a wireless pairing protocol. If the association was unsuccessful (408-N), the process 400 may include error handling (e.g., notifying a user). On the other hand, if the association was successful (408-Y), the process 400 includes adjusting an infusion module (e.g., the infusion device 102) or an infusion pump (e.g., the infusion pump 136) based on the ESD functionality (410). For example, if the ESD functionality includes air-in-line detection, adjusting the infusion module or the infusion pump may include disabling an air-in-line detection function of the infusion module or the infusion pump. For further discussion of how an infusion device may be modified in accordance with an association with an ESD, see the above discussion regarding the process 300.

Turning now to FIG. 4B, the process 400 also includes initiating an infusion therapy with the infusion module (412). For example, initiating the infusion therapy (412) may include initiating the therapy while operating the infusion module or an infusion pump according to an adjusted safety mode, as discussed above with regard to the process 300.

Further, the process 400 includes determining whether the ESD is associated (414). For example, determining whether the ESD is associated (414) may include performing a handshake (e.g., in addition to a handshake performed while initially associating the ESD) with the ESD. As another example, determining whether the ESD is associated (414) may include attempting to send or receive data to or from the ESD.

If it is determined the ESD is no longer associated with the infusion module or the infusion pump (414-N), the process 400 includes continuing the infusion therapy. Moreover, if it is determined that the ESD is no longer associated (414-N), the process 400 may include pausing the infusion therapy or notifying a user that the ESD is no longer associated. An example of this is discussed above with respect to the user interface 260.

However, if it is determined that the ESD is still associated with the infusion module or the infusion pump (414-Y), the process 400 includes determining (416) whether the association is active. For example, an active association may mean that the ESD is actively monitoring (or generating) a safety metric. As another example, an active association may mean that the ESD regularly sends information (e.g., safety metric data, an indication of activity) to the infusion module or the infusion pump.

Responsive to determining that the association is no longer active (416-N), the process 400 includes adjusting the infusion module or the infusion pump to activate on-board safety or pause the infusion (424). Whether an on-board safety mode(s) is activated or the infusion is paused may depend on, for example, the type of drug being infused, patient demographics (e.g., a disease state of the patient), or other infusion parameters. Additionally, examples of other adjustments are discussed above with respect to the process 300. Alternatively, if it is determined that the association is still active (416-Y), the process 400 includes receiving ESD data from the ESD (418). For example, the data may include air-in-line safety metric data (e.g., number of bubbles counted, volume of air detected), or particulate safety metric data (e.g., amount of particulates detected, size of detected particulates, mass of particulates). As another example, the data may include a hazard alert (e.g., indicating an air-in-line hazard, a particulate-in-line hazard).

Furthermore, the process 400 includes generating a safety metric based on infusion pump data (420). In some implementations, the safety metric is generated (420) based on the ESD data, as well. For example, if the ESD includes an air-in-line sensor and the ESD includes air-in-line data, generating the safety metric may include comparing the air-in-line data and air-in-line data gathered by the infusion pump against an air-in-line threshold.

Additionally, the process includes determining whether it is safe to continue the infusion therapy (422). For example, if the safety metric indicates that there is an air-in-line hazard, it may not be safe to continue the infusion therapy. Accordingly, responsive to determining that it is not safe to continue (422-N), the process 400 includes adjusting the infusion module of the infusion pump to activate on-board safety or pause the infusion therapy (424), as discussed above. In addition to the above discussion, activating on-board safety may include triggering an alarm and/or indicating the reason for the alarm (e.g., indicating a type of safety hazard, indicating whether the infusion pump or an ESD detected the safety hazard). However, if it is determined that it is safe to continue the infusion therapy (422-Y), the process 400 includes continuing the infusion therapy (e.g., after ensuring the ESD is still associated, after notifying an operator that the infusion therapy is safe to continue).

Referring back to the associating operation (406), FIG. 4C illustrates an example sub-method of the method 400, particularly with respect to the associating operation (406) and the determination regarding whether the association was successful (408). As illustrated, the associating operation (406) includes associating the infusion module and a fluid (e.g., a medication, a saline solution) with a patient (406A). The associating operation (406) also includes receiving a message identifying an ESD associated with the patient (406B). For example, an ESD may be associated with a patient if a patient has a medical need (e.g., an allergy to a medication) that requires the ESD.

Additionally, the associating operation (406) includes receiving input to associate the ESD with the infusion module and a drug (406C). Further, the associating (406) operation includes determining whether the ESD is connectable with the infusion pump (406D). For example, if the ESD requires a physical (e.g., wired) connection, determining (406D) whether the ESD is connectable with the infusion pump may include determining whether the ESD can be physically connected (e.g., via a particular cable) to the infusion pump. As another example, determining (406D) whether the ESD is connectable with the infusion pump may include determining whether the ESD can wirelessly connect to the infusion pump.

If it is determined that the ESD is not connectable with the infusion pump (406D-N), the associating operation (406) includes receiving a manual confirmation of association (406H). For example, if the ESD operates independently of the infusion pump (e.g., without a handshake, without a pairing), the associating operation (406) may include receiving (e.g., at the infusion pump) a manual confirmation (e.g., from a user) that the ESD is associated with the infusion pump (e.g., monitoring a safety metric of an infusion therapy to be performed by the infusion pump). Alternatively, if it is determined that the ESD is connectable with the infusion pump (406D-Y), the associating operation (406) includes pairing (e.g., physically, wirelessly) the ESD with the infusion pump or the infusion module (406E).

Furthermore, the associating (406) operation includes determining whether the infusion pump can automatically confirm association with the ESD (406F). For example, the infusion pump may be able to automatically confirm association with the ESD via a handshake or data exchange. If it is determined that the infusion pump cannot automatically confirm association with the ESD (406F-N), then the association was unsuccessful (i.e., proceed to 408-N of the process 400). However, if it is determined that the infusion pump can automatically confirm association with the ESD (406F-Y), then the associating operation (406) further includes determining whether the association is confirmed (406G). If the association is confirmed (406G-Y), then the association was successful (i.e., proceed to 408-Y of the process 400); otherwise (406G-N), the association was unsuccessful (i.e., proceed to 408-N).

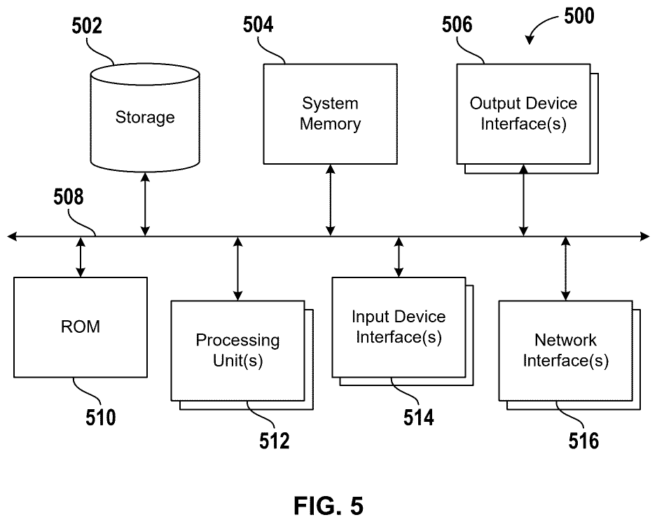

FIG. 5 is a conceptual diagram illustrating an example electronic system 500, according to aspects of the subject technology. The electronic system 500 may be implemented by a computing device for execution of software associated with portions or steps of the processes 300 and 400, or components and methods provided by FIGS. 1-4. In this regard, the electronic system 500 may include infusion device 102. The electronic system 500 may also include a specifically-configured personal computer or a mobile device for infusion such as a smartphone, tablet computer, laptop, PDA, an augmented reality device, a wearable such as a watch or band or glasses, or combination thereof, or other touch screen or television with one or more processors embedded therein or coupled thereto, or any other sort of computer-related electronic device having network connectivity.

The electronic system 500 may also include various types of computer readable media and interfaces for various other types of computer readable media. In the depicted example, the electronic system 500 includes a bus 508, a processing unit(s) 512, a system memory 504, a read-only memory (ROM) 510, a permanent storage device 502, an input device interface(s) 514, an output device interface(s) 506, and a network interface(s) 516. In some implementations, the electronic system 500 may include or be integrated with other computing devices or circuitry for operation of the various components and methods previously described.

The bus 508 collectively represents all system, peripheral, and chipset buses that communicatively connect the numerous internal devices of the electronic system 500. For instance, the bus 508 communicatively connects the processing unit(s) 512 with the ROM 510, the system memory 504, and the permanent storage device 502.

From these various memory units, processing unit(s) 512 retrieves instructions to execute and data to process in order to execute the processes of the subject disclosure. The processing unit(s) 512 can be a single processor or a multi-core processor in different implementations.

The ROM 510 stores static data and instructions that are needed by the processing unit(s) 512 and other modules of the electronic system. The permanent storage device 502, on the other hand, is a read-and-write memory device. This device is a non-volatile memory unit that stores instructions and data even when the electronic system 500 is powered off. Some implementations of the subject disclosure use a mass-storage device (such as a magnetic or optical disk and its corresponding disk drive) as the permanent storage device 502. Other implementations use a removable storage device (such as a floppy disk, flash drive, and its corresponding disk drive) as the permanent storage device 502.

Like the permanent storage device 502, the system memory 504 is a read-and-write memory device. However, unlike the storage device 502, the system memory 504 is a volatile read-and-write memory, such as random-access memory (RAM). The system memory 504 stores some of the instructions and data that the processor needs at runtime. In some implementations, the processes of the subject disclosure are stored in the system memory 504, the permanent storage device 502, and/or the ROM 510. From these various memory units, the processing unit(s) 512 retrieves instructions to execute and data to process, in order to execute the processes of some implementations.

The bus 508 also connects to the input device interface(s) 514 and the output device interface(s) 506. The input device interface(s) 514 enables the user to communicate information and select commands to the electronic system. Input devices used with the input device interface(s) 514 include, for example, alphanumeric keyboards and pointing devices (also called “cursor control devices”). The output device interface(s) 506 enables, for example, the display of images generated by the electronic system 500. Output devices used with the output device interface(s) 506 include, for example, printers and display devices, such as cathode ray tubes (CRT) or liquid crystal displays (LCD). Some implementations include devices (e.g., touchscreens) that function as both input and output devices.

Furthermore, the bus 508 also couples the electronic system 500 to a network (not shown) through the network interface(s) 516. The network interface(s) 516 may include, for example, a wireless access point (e.g., Bluetooth or Wi-Fi) or radio circuitry for connecting to a wireless access point. The network interface(s) 516 may also include hardware (e.g., ethernet hardware) for connecting the computer to a part of a network of computers such as a local area network (LAN), a wide area network (WAN), wireless LAN, an intranet, or a network of networks, such as the Internet. Any or all components of electronic system 500 can be used in conjunction with the subject disclosure when specifically configured with one of more of the features described.

These functions described above can be implemented in computer software, firmware, or hardware. The techniques can be implemented using one or more computer program products. Programmable processors and computers can be included in or packaged as mobile devices. The processes and logic flows can be performed by one or more programmable processors and by programmable logic circuitry. General and special purpose computing devices and storage devices can be interconnected through communication networks.

Some implementations include electronic components, such as microprocessors, storage and memory that store computer program instructions in a machine-readable or computer-readable medium (also referred to as computer-readable storage media, machine-readable media, or machine-readable storage media). Some examples of such computer-readable media include RAM, ROM, read-only compact discs (CD-ROM), recordable compact discs (CD-R), rewritable compact discs (CD-RW), read-only digital versatile discs (e.g., DVD-ROM, dual-layer DVD-ROM), a variety of recordable/rewritable DVDs (e.g., DVD-RAM, DVD-RW, DVD+RW, etc.), flash memory (e.g., SD cards, mini-SD cards, micro-SD cards, etc.), magnetic and/or solid state hard drives, read-only and recordable Blu-Ray® discs, ultra density optical discs, other optical or magnetic media, and floppy disks. The computer-readable media can store a computer program that is executable by at least one processing unit and includes sets of instructions for performing various operations. Examples of computer programs or computer code include machine code, such as is produced by a compiler, and files including higher-level code that are executed by a computer, an electronic component, or a microprocessor using an interpreter.

While the above discussion primarily refers to microprocessor or multi-core processors that execute software, some implementations are performed by one or more integrated circuits, such as application specific integrated circuits (ASICs) or field-programmable gate arrays (FPGAs). In some implementations, such integrated circuits execute instructions that are stored on the circuit itself.

As used in this specification and any claims of this application, the terms “computer,” “server,” “processor,” and “memory” all refer to electronic or other technological devices specifically configured with one or more of the features described above. These terms exclude people or groups of people. For the purposes of the specification, the terms display or displaying means displaying on an electronic device. As used in this specification and any claims of this application, the terms “computer readable medium” and “computer readable media” are entirely restricted to tangible, physical objects that store information in a form that is readable by a computer. These terms exclude any wireless signals, wired download signals, and any other ephemeral signals.

To provide for interaction with a user, implementations of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well. For example, feedback provided to the user can be any form of sensory feedback (e.g., visual feedback, auditory feedback, tactile feedback), and input from the user can be received in forms such as acoustic, speech, gesture, or tactile input. In addition, a computer can interact with a user by sending documents to and receiving documents from a device that is used by the user (e.g., by sending web pages to a web browser on a user's client device in response to requests received from the web browser).

Implementations of the subject matter described in this specification can be implemented in a specifically configured computing system that includes a back end component (e.g., a data server), or that includes a specifically configured middleware component (e.g., an application server), or that includes a specifically configured front end component (e.g., a client computer having a graphical user interface or a Web browser through which a user can interact with an implementation of the subject matter described in this specification), or any combination of one or more such back end, middleware, or front end components. The components of the system can be interconnected by one or more forms or mediums of digital data communication, such as a communication network. Examples of communication networks include a LAN and a WAN, an inter-network (e.g., the Internet), and peer-to-peer networks (e.g., ad hoc peer-to-peer networks).

The computing system can include specifically configured clients and servers. A client and server are generally remote from each other and may interact through a communication network. The relationship of client and server arises by virtue of computer programs running on the respective computers and having a client-server relationship to each other. In some implementations, a server transmits data (e.g., an HTML page) to a client device (e.g., for purposes of displaying data to and receiving user input from a user interacting with the client device). Data generated at the client device (e.g., a result of the user interaction) can be received from the client device at the server.

Those of skill in the art will appreciate that the various illustrative blocks, modules, elements, components, methods, and algorithms described herein may be implemented as electronic hardware, computer software, or a combination thereof. To illustrate this interchangeability of hardware and software, various illustrative blocks, modules, elements, components, methods, and algorithms have been described above generally in terms of their functionality. Whether such functionality is implemented as hardware or software depends upon the particular application and design constraints imposed on the overall system. The described functionality may be implemented in varying ways for each particular application. Various components and blocks may be arranged differently (e.g., arranged in a different order, or partitioned in a different way) all without departing from the scope of the subject technology.

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order and are not meant to be limited to the specific order or hierarchy presented.

Illustration of Subject Technology as Clauses:

Various examples of aspects of the disclosure are described as numbered clauses (1, 2, 3, etc.) for convenience. These are provided as examples, and do not limit the subject technology. Identifications of the figures and reference numbers are provided below merely as examples and for illustrative purposes, and the clauses are not limited by those identifications.

Clause 1. An infusion device, comprising: a safety metric sensor; a processor; and a non-transitory, computer-readable storage medium storing instructions that, when executed by the processor, cause the infusion device to: operate in a default safety mode, wherein operating in the default safety mode comprises monitoring, via the safety metric sensor, an infusion therapy for compliance with a safety metric indicating a property of a fluid path between a fluid container and a fluid administration site; connect, while operating in the default safety mode, with an external safety device (“ESD”) that is configured to monitor the safety metric; determine, based on the connection with the ESD, that the ESD is monitoring the safety metric; and switch, responsive to determining that the ESD is monitoring the safety metric, from operating in the default safety mode to operating in an adjusted safety mode, wherein operating in the adjusted safety mode comprises: pausing monitoring the infusion therapy for compliance with the safety metric via the safety metric sensor; or monitoring the infusion therapy for compliance with the safety metric via the ESD.

Clause 2. The infusion device of Clause 1, wherein the non-transitory, computer-readable storage medium further stores instructions that, when executed by the processor, cause the infusion device to: determine, based on the connection (e.g., a lack of a connection) with the ESD, that the ESD is no longer monitoring the safety metric; and switch, responsive to determining that the ESD is no longer monitoring the safety metric, from operating in the adjusted safety mode to operating in the default safety mode.

Clause 3. The infusion device of any one of Clauses 1 through 2, wherein the non-transitory, computer-readable medium further stores instructions that, when executed by the processor, cause the infusion device to: receive an infusion parameter; determine, based on a characteristic of the infusion parameter, that the infusion therapy should be monitored via the ESD; and prompt a user of the infusion device to connect the ESD to the infusion device.

Clause 4. The infusion device of any one of Clauses 1 through 3, wherein operating in the adjusted safety mode comprises: monitoring the infusion therapy for compliance with the safety metric; collecting safety metric data via the safety metric sensor; receiving additional safety metric data from the ESD; and determining whether the safety metric satisfies a hazard threshold based on the safety metric data and the additional safety metric data.

Clause 5. The infusion device of Clause 4, wherein operating in the adjusted safety mode further comprises, responsive to determining that the safety metric satisfies the hazard threshold: pausing an infusion therapy; or activating a safety mode of the infusion device.

Clause 6. The infusion device of any one of Clauses 1 through 5, wherein: operating in the default safety mode comprises triggering an alarm in response to detecting that the safety metric satisfies a hazard threshold; and operating in the adjusted safety mode comprises deactivating the alarm.

Clause 7. The infusion device of any one of Clauses 1 through 5 wherein: operating in the default safety mode comprises triggering an alarm in response to detecting that the safety metric satisfies a hazard threshold; and operating in the adjusted safety mode comprises triggering an alarm in response to detecting that the safety metric satisfies an adjusted hazard threshold that is different from the hazard threshold.

Clause 8. The infusion device of any one of Clauses 1 through 7, wherein: operating in the default safety mode comprises causing the safety metric sensor to sample the safety metric at a default sample rate; and operating in the adjusted safety mode comprises causing the safety metric sensor to sample the safety metric at an adjusted sample rate that is different from the default sample rate.

Clause 9. The infusion device of any one of Clauses 1 through 8, wherein the non-transitory, computer-readable storage medium further stores instructions that, when executed by the processor, cause the infusion device to: receive an infusion request indicating an infusion therapy and an ESD requirement that specifies a type of ESD; receive a type of the ESD from the ESD; determine that the type of the ESD matches the specified type of ESD; and initiate the infusion therapy responsive to determining that the type of the ESD matches the specified type of ESD.

Clause 10. The infusion device of any one of Clauses 1 through 9, wherein: the ESD comprises an air trap, an air detector, a flow detector, a particulate detector, or a spectral analyzer; the safety metric comprises an air-in-line metric, a flow metric, a particulate-in-line metric, or a spectral metric; and determining that the ESD is monitoring the safety metric comprises receiving, from the ESD, additional safety metric data comprising air-in-line data, flow data, particulate-in-line data, or spectral metric data.

Clause 11. A computer-implemented method, comprising: activating an infusion device and a safety metric sensor; operating the infusion device in a default safety mode, wherein operating in the default safety mode comprises monitoring, via the safety metric sensor, an infusion therapy for compliance with a safety metric indicating a property of a fluid path between a fluid container and a fluid administration site; connecting, while operating the infusion device in the default safety mode, the infusion device with an external safety device (“ESD”) that is configured to monitor the safety metric; determining, based on the connection with the ESD, that the ESD is monitoring the safety metric; and switching the infusion device, responsive to determining that the ESD is monitoring the safety metric, from operating in the default safety mode to operating in an adjusted safety mode, wherein operating in the adjusted safety mode comprises: pausing monitoring the infusion therapy for compliance with the safety metric via the safety metric sensor; or monitoring the infusion therapy for compliance with the safety metric via the ESD.

Clause 12. The computer-implemented method of Clause 11, further comprising: determining, based on the connection (e.g., a lack of a connection) between the infusion device and the ESD, that the ESD is no longer monitoring the safety metric; and switching the infusion device, responsive to determining that the ESD is no longer monitoring the safety metric, from operating in the adjusted safety mode to operating in the default safety mode.

Clause 13. The computer-implemented method of any one of Clauses 11 through 12, further including: receiving an infusion parameter; determining, based on a characteristic of the infusion parameter, that the infusion therapy should be monitored via the ESD; and prompting a user of the infusion device to connect the ESD to the infusion device.

Clause 14. The computer-implemented method of any one of Clauses 11 through 13, wherein operating in the adjusted safety mode comprises: monitoring the infusion therapy for compliance with the safety metric; collecting safety metric data via the safety metric sensor; receiving additional safety metric data from the ESD; and determining whether the safety metric satisfies a hazard threshold based on the safety metric data and the additional safety metric data.

Clause 15. The computer-implemented method of Clause 14, wherein operating in the adjusted safety mode further comprises, responsive to determining that the safety metric satisfies the hazard threshold: pausing an infusion therapy; or activating a safety mode of the infusion device.

Clause 16. The computer-implemented method of any one of Clauses 11 through 15, wherein: operating in the default safety mode comprises triggering an alarm in response to detecting that the safety metric satisfies a hazard threshold; and operating in the adjusted safety mode comprises deactivating the alarm.

Clause 17. The computer-implemented method of any one of Clauses 11 through 15, wherein: operating in the default safety mode comprises triggering an alarm in response to detecting that the safety metric satisfies a hazard threshold; and operating in the adjusted safety mode comprises triggering an alarm in response to detecting that the safety metric satisfies an adjusted hazard threshold that is different from the hazard threshold.

Clause 18. The computer-implemented method of any one of Clauses 11 through 17, wherein: operating in the default safety mode comprises causing the safety metric sensor to sample the safety metric at a default sample rate; and operating in the adjusted safety mode comprises causing the safety metric sensor to sample the safety metric at an adjusted sample rate that is different from the default sample rate.

Clause 19. The computer-implemented method of any one of Clauses 11 through 18, further comprising: receiving, at the infusion device, an infusion request indicating an infusion therapy and an ESD requirement that specifies a type of ESD; receiving, at the infusion device, a type of the ESD from the ESD; determining that the type of the ESD matches the specified type of ESD; and initiating the infusion therapy at the infusion device responsive to determining that the type of the ESD matches the specified type of ESD.

Clause 20. The computer-implemented method of any one of Clauses 11 through 19, wherein: the ESD comprises an air trap, an air detector, a flow detector, a particulate detector, or a spectral analyzer; the safety metric comprises an air-in-line metric, a flow metric, a particulate-in-line metric, or a spectral metric; and determining that the ESD is monitoring the safety metric comprises receiving, from the ESD, additional safety metric data comprising air-in-line data, flow data, particulate-in-line data, or spectral metric data.

### Further Consideration

It is understood that the specific order or hierarchy of steps in the processes disclosed is an illustration of example approaches. Based upon design preferences, it is understood that the specific order or hierarchy of steps in the processes may be rearranged. Some of the steps may be performed simultaneously. The accompanying method claims present elements of the various steps in a sample order, and are not meant to be limited to the specific order or hierarchy presented.

The previous description is provided to enable any person skilled in the art to practice the various aspects described herein. The previous description provides various examples of the subject technology, and the subject technology is not limited to these examples. Various modifications to these aspects will be readily apparent to those skilled in the art, and the generic principles defined herein may be applied to other aspects.

Thus, the claims are not intended to be limited to the aspects shown herein but are to be accorded the full scope consistent with the language of the claims. For example, reference to an element in the singular is not intended to mean “one and only one” unless specifically so stated, but rather “one or more.” Moreover, unless specifically stated otherwise, the term “some” refers to one or more. Pronouns in the masculine (e.g., his) include the feminine and neuter gender (e.g., her and its) and vice versa. Headings and subheadings, if any, are used for convenience only and do not limit the invention described herein.

The predicate words “configured to,” “operable to,” and “programmed to” do not imply any particular tangible or intangible modification of a subject, but, rather, are intended to be used interchangeably. For example, a processor configured to monitor and control an operation or a component, may also mean the processor being programmed to monitor and control the operation or the processor being operable to monitor and control the operation. Likewise, a processor configured to execute code can be construed as a processor programmed to execute code or operable to execute code.

The term “automatic,” as used herein, may include performance by a computer or machine without user intervention, for example, by instructions responsive to a predicate action by the computer or machine or other initiation mechanism. The word “example” is used herein to mean “serving as an example or illustration.” Any aspect or design described herein as “example” is not necessarily to be construed as preferred or advantageous over other aspects or designs.

A phrase such as an “aspect” does not imply that such aspect is essential to the subject technology or that such aspect applies to all configurations of the subject technology. A disclosure relating to an aspect may apply to all configurations, or one or more configurations. An aspect may provide one or more examples. A phrase such as an aspect may refer to one or more aspects and vice versa. A phrase such as an “implementation” does not imply that such implementation is essential to the subject technology or that such implementation applies to all configurations of the subject technology. A disclosure relating to an implementation may apply to all implementations, or one or more implementations. An implementation may provide one or more examples. A phrase such as an “implementation” may refer to one or more implementations and vice versa. A phrase such as a “configuration” does not imply that such configuration is essential to the subject technology or that such configuration applies to all configurations of the subject technology. A disclosure relating to a configuration may apply to all configurations, or one or more configurations. A configuration may provide one or more examples. A phrase such as a “configuration” may refer to one or more configurations and vice versa.

As used herein a “user interface” (also referred to as an interactive user interface, a graphical user interface, or a UI) may refer to a network-based interface including data fields or other control elements for receiving input signals or providing electronic information or for providing information to the user in response to any received input signals. Control elements may include dials, buttons, icons, selectable areas, or other perceivable indicia presented via the UI that, when interacted with (e.g., clicked, touched, selected, etc.), initiates an exchange of data for the device presenting the UI. A UI may be implemented in whole or in part using technologies such as hyper-text mark-up language (HTML), FLASH™, JAVA™, .NET™, C, C++, web services, or rich site summary (RSS). In some implementations, a UI may be included in a stand-alone client (for example, thick client, fat client) configured to communicate (e.g., send or receive data) in accordance with one or more of the aspects described. The communication may be to or from a medical device or server in communication therewith.

As used herein, the terms “determine” or “determining” encompass a wide variety of actions. For example, “determining” may include calculating, computing, processing, deriving, generating, obtaining, looking up (e.g., looking up in a table, a database, or another data structure), ascertaining and the like via a hardware element without user intervention. Also, “determining” may include receiving (e.g., receiving information), accessing (e.g., accessing data in a memory) and the like via a hardware element without user intervention. “Determining” may include resolving, selecting, choosing, establishing, and the like via a hardware element without user intervention.

As used herein, the terms “provide” or “providing” encompass a wide variety of actions. For example, “providing” may include storing a value in a location of a storage device for subsequent retrieval, transmitting a value directly to the recipient via at least one wired or wireless communication medium, transmitting or storing a reference to a value, and the like. “Providing” may also include encoding, decoding, encrypting, decrypting, validating, verifying, and the like via a hardware element.

As used herein, the term “message” encompasses a wide variety of formats for communicating (e.g., transmitting or receiving) information. A message may include a machine-readable aggregation of information such as an XML document, fixed field message, comma separated message, JSON, a custom mode, or the like. A message may, in some implementations, include a signal utilized to transmit one or more representations of the information. While recited in the singular, it will be understood that a message may be composed, transmitted, stored, received, etc. in multiple parts.

As used herein, the term “selectively” or “selective” may encompass a wide variety of actions. For example, a “selective” process may include determining one option from multiple options. A “selective” process may include one or more of: dynamically determined inputs, preconfigured inputs, or user-initiated inputs for making the determination. In some implementations, an n-input switch may be included to provide selective functionality where n is the number of inputs used to make the selection.

As used herein, the terms “correspond” or “corresponding” encompasses a structural, functional, quantitative and/or qualitative correlation or relationship between two or more objects, data sets, information and/or the like, preferably where the correspondence or relationship may be used to translate one or more of the two or more objects, data sets, information and/or the like so to appear to be the same or equal. Correspondence may be assessed using one or more of a threshold, a value range, fu5y logic, pattern matching, a machine learning assessment model, or combinations thereof.

In some implementations, data generated or detected can be forwarded to a “remote” device or location, where “remote,” means a location or device other than the location or device at which the program is executed. For example, a remote location could be another location (e.g., office, lab, etc.) in the same city, another location in a different city, another location in a different state, another location in a different country, etc. As such, when one item is indicated as being “remote” from another, what is meant is that the two items can be in the same room but separated, or at least in different rooms or different buildings, and can be at least one mile, ten miles, or at least one hundred miles apart. “Communicating” information references transmitting the data representing that information as electrical signals over a suitable communication channel (e.g., a private or public network). “Forwarding” an item refers to any means of getting that item from one location to the next, whether by physically transporting that item or otherwise (where that is possible) and includes, at least in the case of data, physically transporting a medium carrying the data or communicating the data. Examples of communicating media include radio or infra-red transmission channels as well as a network connection to another computer or networked device, and the internet or including email transmissions and information recorded on websites and the like.

1. An infusion device, comprising:
a safety metric sensor;
a processor; and
a non-transitory, computer-readable storage medium storing instructions that, when executed by the processor, cause the infusion device to:
operate in a default safety mode, wherein operating in the default safety mode comprises monitoring, via the safety metric sensor, an infusion therapy for compliance with a safety metric indicating a property of a fluid path between a fluid container and a fluid administration site;
connect, while operating in the default safety mode, with an external safety device (“ESD”) that is configured to monitor the safety metric;
determine, based on the connection with the ESD, that the ESD is monitoring the safety metric; and
switch, responsive to determining that the ESD is monitoring the safety metric, from operating in the default safety mode to operating in an adjusted safety mode, wherein operating in the adjusted safety mode comprises:
pausing monitoring the infusion therapy for compliance with the safety metric via the safety metric sensor; or
monitoring the infusion therapy for compliance with the safety metric via the ESD.

2. The infusion device of claim 1, wherein the non-transitory, computer-readable storage medium further stores instructions that, when executed by the processor, cause the infusion device to:
determine, based on the connection with the ESD, that the ESD is no longer monitoring the safety metric; and
switch, responsive to determining that the ESD is no longer monitoring the safety metric, from operating in the adjusted safety mode to operating in the default safety mode.

3. The infusion device of claim 1, wherein the non-transitory, computer-readable medium further stores instructions that, when executed by the processor, cause the infusion device to:
receive an infusion parameter;
determine, based on a characteristic of the infusion parameter, that the infusion therapy should be monitored via the ESD; and
prompt a user of the infusion device to connect the ESD to the infusion device.

4. The infusion device of claim 1, wherein operating in the adjusted safety mode comprises:
monitoring the infusion therapy for compliance with the safety metric;
collecting safety metric data via the safety metric sensor of the infusion device;
receiving additional safety metric data from the ESD; and
determining whether the safety metric satisfies a hazard threshold based on the safety metric data and the additional safety metric data.

5. The infusion device of claim 4, wherein operating in the adjusted safety mode further comprises, responsive to determining that the safety metric satisfies the hazard threshold:
pausing an infusion therapy; or activating a safety mode of the infusion device.

6. The infusion device of claim 1, wherein:
operating in the default safety mode comprises triggering an alarm in response to detecting that the safety metric satisfies a hazard threshold; and
operating in the adjusted safety mode comprises deactivating the alarm.

7. The infusion device of claim 1, wherein:
operating in the default safety mode comprises triggering an alarm in response to detecting that the safety metric satisfies a hazard threshold; and
operating in the adjusted safety mode comprises triggering an alarm in response to detecting that the safety metric satisfies an adjusted hazard threshold that is different from the hazard threshold.

8. The infusion device of claim 1, wherein:
operating in the default safety mode comprises causing the safety metric sensor of the infusion device to sample the safety metric at a default sample rate; and
operating in the adjusted safety mode comprises causing the safety metric sensor to sample the safety metric at an adjusted sample rate that is different from the default sample rate.

9. The infusion device of claim 1, wherein the non-transitory, computer-readable storage medium further stores instructions that, when executed by the processor, cause the infusion device to:
receive an infusion request indicating an infusion therapy and an ESD requirement that specifies a type of ESD;
receive a type of the ESD from the ESD;
determine that the type of the ESD matches the specified type of ESD; and
initiate the infusion therapy responsive to determining that the type of the ESD matches the specified type of ESD.

10. The infusion device of claim 1, wherein:
the ESD comprises an air trap, an air detector, a flow detector, a particulate detector, or a spectral analyzer;
the safety metric comprises an air-in-line metric, a flow metric, a particulate-in-line metric, or a spectral metric; and
determining that the ESD is monitoring the safety metric comprises receiving, from the ESD, additional safety metric data comprising air-in-line data, flow data, particulate-in-line data, or spectral metric data.

11. A computer-implemented method, comprising:
activating an infusion device and a safety metric sensor;
operating the infusion device in a default safety mode, wherein operating in the default safety mode comprises monitoring, via the safety metric sensor, an infusion therapy for compliance with a safety metric indicating a property of a fluid path between a fluid container and a fluid administration site;
connecting, while operating the infusion device in the default safety mode, the infusion device with an external safety device (“ESD”) that is configured to monitor the safety metric;
determining, based on the connection with the ESD, that the ESD is monitoring the safety metric; and
switching the infusion device, responsive to determining that the ESD is monitoring the safety metric, from operating in the default safety mode to operating in an adjusted safety mode, wherein operating in the adjusted safety mode comprises:
pausing monitoring the infusion therapy for compliance with the safety metric via the safety metric sensor; or
monitoring the infusion therapy for compliance with the safety metric via the ESD.

12. The computer-implemented method of claim 11, further comprising:
determining, based on the connection between the infusion device and the ESD, that the ESD is no longer monitoring the safety metric; and
switching the infusion device, responsive to determining that the ESD is no longer monitoring the safety metric, from operating in the adjusted safety mode to operating in the default safety mode.

13. The computer-implemented method of claim 11, further including:
receiving an infusion parameter;
determining, based on a characteristic of the infusion parameter, that the infusion therapy should be monitored via the ESD; and
prompting a user of the infusion device to connect the ESD to the infusion device.

14. The computer-implemented method of claim 11, wherein operating in the adjusted safety mode comprises:
monitoring the infusion therapy for compliance with the safety metric;
collecting safety metric data via the safety metric sensor;
receiving additional safety metric data from the ESD; and
determining whether the safety metric satisfies a hazard threshold based on the safety metric data and the additional safety metric data.

15. The computer-implemented method of claim 14, wherein operating in the adjusted safety mode further comprises, responsive to determining that the safety metric satisfies the hazard threshold:
pausing an infusion therapy; or
activating a safety mode of the infusion device.

16. The computer-implemented method of claim 11, wherein:
operating in the default safety mode comprises triggering an alarm in response to detecting that the safety metric satisfies a hazard threshold; and
operating in the adjusted safety mode comprises deactivating the alarm.

17. The computer-implemented method of claim 11, wherein:
operating in the default safety mode comprises triggering an alarm in response to detecting that the safety metric satisfies a hazard threshold; and
operating in the adjusted safety mode comprises triggering an alarm in response to detecting that the safety metric satisfies an adjusted hazard threshold that is different from the hazard threshold.

18. The computer-implemented method claim 11, wherein:
operating in the default safety mode comprises causing the safety metric sensor to sample the safety metric at a default sample rate; and
operating in the adjusted safety mode comprises causing the safety metric sensor to sample the safety metric at an adjusted sample rate that is different from the default sample rate.

19. The computer-implemented method of claim 11, further comprising:
receiving, at the infusion device, an infusion request indicating an infusion therapy and an ESD requirement that specifies a type of ESD;
receiving, at the infusion device, a type of the ESD from the ESD;
determining that the type of the ESD matches the specified type of ESD; and
initiating the infusion therapy at the infusion device responsive to determining that the type of the ESD matches the specified type of ESD.

20. The computer-implemented method of claim 11, wherein:
the ESD comprises an air trap, an air detector, a flow detector, a particulate detector, or a spectral analyzer;
the safety metric comprises an air-in-line metric, a flow metric, a particulate-in-line metric, or a spectral metric; and
determining that the ESD is monitoring the safety metric comprises receiving, from the ESD, additional safety metric data comprising air-in-line data, flow data, particulate-in-line data, or spectral metric data.
